(19) United States
(12) Patent Application Publication
(10) Pub. No.: US 2003/0007964 A1
(43) Pub. Date: Jan. 9, 2003

(54) Method for treating rheumatoid arthritis with composition containing histone

(75) Inventors: Insoo Bae, Daejon (KR); Dong-soo Kim, Seoul (KR); Heajoon Yim, Daejon (KR); Neon-Cheol Jung, Daejon (KR); Yong-Weon Yi, Daejon (KR); Seung-Suh Hong, Daejon (KR); Hyun-Soo Lee, Seoul (KR)

Correspondence Address: DARBY & DARBY P.C., Post office Box 5257, New York, NY, 10150-5257, US

(73) Assignee: SAMYANG GENEX CORPORATION

(21) Appl. No.: 10/171,388

(22) Filed: Jun. 12, 2002

Related U.S. Application Data
(63) Continuation of application No. 09/803,171, filed on Dec. 18, 2000.
Application No. 09/803,171 is a continuation-in-part of application No. 09/381,559, filed on Jan. 19, 2000, now Pat. No. 6,204,242.
Application No. 09/381,559 is a 371 of international application No. PCT/KR99/00037, filed on Jan. 19, 2000.

(30) Foreign Application Priority Data
Jan. 24, 1998 (KR) 1998/3362

(51) Int. Cl. A61K 38/47

(52) U.S. Cl. 424/94.61

(57) ABSTRACT

The present invention relates to a novel use of histone and an active fragment thereof and provides a pharmaceutical composition containing histone as an active ingredient to improve the symptoms of progressive, inflammatory and autoimmune arthritis. The pharmaceutical composition of the present invention includes histone, especially histone H1, histone H2A, histone H2B, histone H3, histone H4, an active fragment thereof, and a mixture thereof as an active ingredient, and could include pharmacologically approved carriers if necessary. Histone or its active fragment lowered induction of arthritis and reduced arthritis index more effectively than steroidal dexamethasone and also had a significant preventive effect.

### TECHNICAL FIELD

The present invention relates to the use of histone or an active fragment thereof in improving inflammatory symptoms of arthritis by many possible mechanisms. Histone or an active fragment thereof lowers induction of arthritis and reduces the arthritis index more effectively than steroidal dexamethasone and also has a significant preventive effect.

### BACKGROUND OF THE INVENTION

The present invention relates to biologically active compositions to improve symptoms of progressive, inflammatory and autoimmune arthritis. Despite the development of many arthritis drugs, arthritis remains a world wide serious disease due to an increasing aging population. Even though the death rate due to arthritis is low, the quality of life of an individual who suffers from this disease is sacrificed with lowered activity level and productivity.

Among many types of arthritis, the most significant one is rheumatoid arthritis. Rheumatoid arthritis is an autoimmune disease caused by the action of auto-reactive T lymphocytes. T lymphocytes cause rheumatoid arthritis via secondary hypersensitivity. It is not fully understood which antigen is recognized by T lymphocytes to cause this disease. Type II collagen is known to be the most probable one, but other possibilities cannot be excluded. Anti-histone autoantibody has been discovered even though it is not clear that this antibody is the cause of the disease.

Many drugs have been used to treat rheumatoid arthritis without complete relief of the symptoms. Conventional drugs include non-steroidal anti-inflammatory drugs (NSAIDs, aspirin, ibuprofen), gold salt, penicilamine, and steroidal hormones. The most potent and effective steroidal hormones have side effects when taken orally for a long period. Recently, water-soluble ligands of tumor necrosis factor (TNF), that play a major role in the inflammation mechanism, were produced by using recombinant gene technology to treat this disease. However, an improved formulation to treat symptoms of rheumatoid arthritis such as inflammation, edema, abnormal formation of new blood vessels, destruction of cartilage and bone erosion is required.

Collagen-induced arthritis (CIA) has been used as an animal model of the T-lymphoidal rheumatoid arthritis (Autoimmunity to Type II collagen: Experimental model of arthritis, J. Exp. Med. 146; 857-868 (1977)). When type II collagen was injected into mice, which are prone to develop arthritis, arthritis was induced within 2 weeks with symptoms such as formation of pannus, erosion of cartilage and bone. Like rheumatoid arthritis, CIA also has humoral and the cellular immune responses against collagen.

Histone is one of the major nuclear components in the cells and forms chromosomes with nucleic acids. Many different forms of histones were isolated from mammals other than humans. There are many reports regarding various physiological activities of histone H1, H2A, and the like.

The discovery and isolation of water-soluble histone H1 in bovine plasma and milk was reported in Biochem. J. Vol. 244, 675-682, 1987. Proc. Natl. Acad. Sci. USA vol. 82, 4871-4875, which reported that the major component of the homeostatic thymus hormone (HTH) is histone H1. Histone H1 circulates freely in the lymph and blood vessels and acts similarly to hormones such as by controlling the secretion of other hormones.

Ann. J. Med. Sci. vol. 250, 79-85, 1965 also reported that the HTH therapy could potentiate the immune system and resolve the immunological problems associated with thymectomy. WO 8503003A suggests using histone H1 fragment, which has the characteristics of thymus hormone, as an immunotherapy to prevent leukemia after thymectomy or radiotherapy of thymus. U.S. Pat. No. 5,182,257 disclosed histones H1, H2A, H2B, H3 and H4 as drugs for lymphoma or leukemia.

Chemical abstracts 74,85743 (1971) reported that, when taken together with T2 bacteriophage, histone H1 subunit could down-regulate the formation of antibodies against T2 bacteriophage. Chemical abstracts 73,96837 (1970) reported the use of histone H1 as an immunosuppressant for skin grafting.

Nature vol. 360, 33-39, 1992 reported that histone H1 can stabilize the flagellar microtubule structure of sea urchin. J. Biol. Chem vol. 259,15523-15531, 1984 reported that histone H1, acting with the microtubules isolated from murine brain, induces aggregation of tubulin which is similar to the ring structure of the microtubules.

The discovery and isolation of histone H2A in various mammals including human was reported in Nuc. Acids Res. Vol 17, p317-346, 1989.

Proc. Natl. Acad. Sci USA 1985;82:4871-5 reported that histones H2A and H2B form a stable dimer(H2A/H2B) and both of them are components of homeostatic thymus hormone which can be used in treatment of arthritis.

No existing references, however, suggest using histone or an active fragment thereof as a drug to treat rheumatoid arthritis.

### BRIEF DESCRIPTION OF THE DRAWINGS

FIG. 1 is a graph showing the changes in the induction of rheumatoid arthritis after administration of histone H1 (● . . . ●: no treatment after collagen inoculation (control group), [symbol] ______ [symbol] : a group that had dexamethasone for a preventive effect, [symbol] . . . [symbol] : a group that had dexamethasone for a treatment effect, ▪ ______▪: the group that had histone H1 for a preventive effect,: ▪ . . . ▪: a group that had histone H1 for a treatment effect).

FIG. 2A is a graph showing the preventive effect of histone H1 against rheumatoid arthritis (● ______●: no treatment after collagen inoculation (control group), [symbol] ______ [symbol] : the group that had dexamethasone injection, ● ______●: a group that had histone H1 injection).

FIG. 2B is a graph showing the treatment effect of histone H1 against rheumatoid arthritis by the changes in arthritis index with time (● ______●: no treatment after collagen inoculation (control group), [symbol] ______ [symbol] : the group that had dexamethasone treatment, ▪ ______▪: the group that had histone H1 treatment).

FIG. 3A is a picture of a fore leg of a mouse in the control group showing edema at 6 weeks after collagen inoculation.

FIG. 3B is a picture of a fore leg of a mouse that had histone H1 administration at 6 weeks after collagen inoculation.

FIGS. 4A and 4B are the sections of knee joints of control group mice showing the formation of pannus, destruction of cartilage, bone erosion and manifestation of inflammatory cells at 10 weeks after the collagen antigen inoculation (P=pannus, C=cartilage, J=joint space).

FIG. 4C is a section of knee joint of a test group mouse that had histone H1 treatment at 10 weeks after the collagen antigen inoculation (P=pannus, C=cartilage, J=joint space).

FIG. 5 is a graph showing the treatment effect of histone H2A against rheumatoid arthritis by the changes in arthritis index with time (●- ●: no treatment after collagen inoculation (control group), ◯ - ◯: the group that had dexamethasone treatment, Δ-Δ: the group that had histone H2A treatment).

FIG. 6 is a graph showing the treatment effect of histone H2An active fragment against rheumatoid arthritis by the changes in arthritis index with time (●- ●: no treatment after collagen inoculation (control group), ▴ - ▴: the group that had histone H2An active fragment treatment).

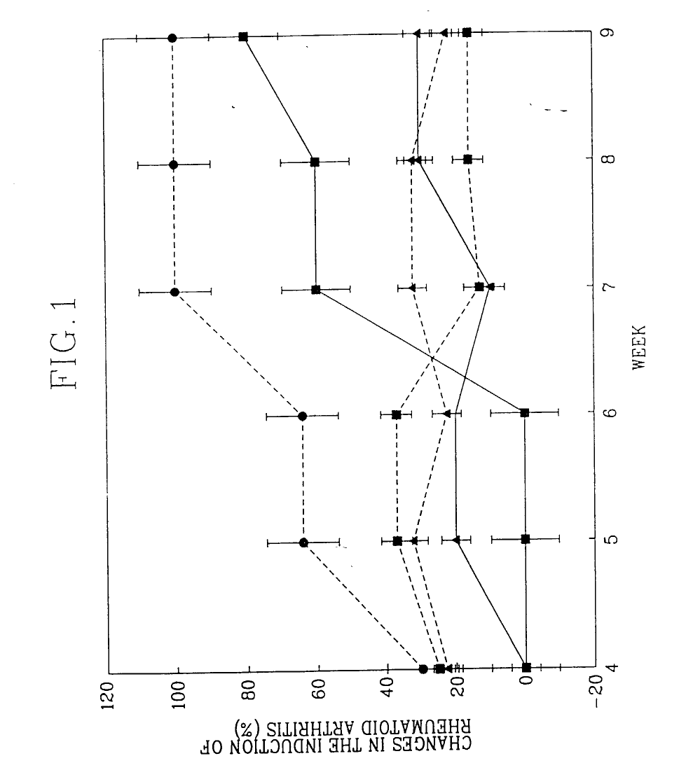

### SUMMARY OF THE INVENTION

It is an object of the present invention to provide a pharmaceutical composition that is more effective than conventional formulations to improve the symptoms of progressive, inflammatory and autoimmune arthritis.

It is an other object of the present invention to provide a pharmaceutical composition containing histone or an active fragment thereof to release the symptoms of progressive, inflammatory and autoimmune arthritis.

It is a further object of the present invention to provide a pharmaceutical composition containing histone or an active fragment thereof to prevent the invasion, of progressive, inflammatory and autoimmune arthritis.

### DETAILED DESCRIPTION OF THE INVENTION

The present invention relates to a novel use of histone and provides a pharmaceutical composition containing histone as an active ingredient to improve symptoms of progressive, inflammatory and autoimmune arthritis.

The symptomatic alleviation includes 1) the improvement of arthritis related symptoms; 2) the prevention of the progress in a progressive disease; and 3) the prevention of invasion in an arthritis prone individual.

The pharmaceutical composition of the present invention includes histone, especially histone H1, histone H2A, histone H2B, histone H3, histone H4, an active fragment of the foregoing, or a mixture thereof as an active ingredient, and may include pharmacologically approved carriers if necessary.

The pharmaceutical composition of the present invention may be used by itself or in combination with conventional drugs for arthritis.

To determine that the symptoms of autoimmune rheumatoid arthritis could be alleviated by the histone treatment, histone or an active fragment thereof was administered to mammals that were invaded by or prone to arthritis. Collagen induced arthritis, which is a well-known animal model for rheumatoid arthritis, was induced in experimental mice.

In the present invention, mammals can be extended to human and arthritis can be extended into rheumatoid arthritis. There is no limitation in the origin to isolate histone in the present invention. Also, histone can be extended into histone H2A, H2B, H3 and H4, active fragment thereof or a mixture thereof.

In the present specification, “active fragment” means any fragment derived from histone which is effective to release or treat the symptoms of arthritis.

The required amount of histone or active fragment thereof enough to prevent the symptoms of arthritis is ca. 1 mg/kg body weight.

In the present invention, the interval of administration was 3-4 days, but the interval can be extended to 1, 2 or 4 weeks. The ideal means of administration is intravenous or intraperitoneal injection, but other methods can also be used.

The most effective administration, the amount and the interval of administration could be controlled with ease by observing the degree of symptomatic progress or the reaction of the patient after administration according to the diagnosis or the prescription of a doctor.

The invention will be illustrated further by the following examples, but not limited to the examples given.

To estimate the average values in each experimental group, Student s t-test was used in the examples of the present invention. Chi-square test was used to estimate the standard deviation. The result was considered statistically significant when p<0.05.

### EXAMPLE 1

### Induction of Rheumatoid Arthritis in Experimental Mice

Induction of Arthritis

Five-week old DBA/1J female mice were imported from Charles River Japan and allowed to adapt in an animal room for two weeks before using them in the experiments at the age of 7 weeks (20-25 g).

Isolated and quantified type II collagen of chicken (Sigma Chemical Co., St. Louis, Mo., U.S.A.) was solubilized in 0.1 N acetic acid at a concentration of 2 mg/ml. The solution was mixed with an equal amount of a complete Freund's adjuvant at 4° C. to form a suspension. One hundred microliters of this mixture was injected intravenously around the origin of the tail vein and further inoculated at 3 and 6 weeks after the first injection (D. E. Trentham et. al., Autoimmunity to Type II collagen: An Experimental Model of Arthritis, J. Exp. Med. 146; 857-868 (1977)). Arthritis was induced from the 4^{th} week after the first injection.

Estimation of Arthritis

Clinical incidence of arthritis % (C.I.A) and arthritis index were examined. C.I.A. was expressed as the percentage of mice that have arthritic symptoms among the total mice. The degree of inflammation expressed as the arthritis index was categorized from 0 to 3 by 2 researchers every week as below. Pictures of the feet of some mice were taken 6 weeks after the collagen administration.

0: normal condition

1: One or two toes were swollen with suffusion or had minimal swelling

2: Definite suffusion or local swelling in many toes

3: Observation of severe edema and swelling up to the knee and restricted use of the feet.

The arthritis index was calculated for 4 feet (2 hind feet and 2 fore feet) giving the maximum value of 12. An index of 6-8 was considered severe since collagen induced arthritis invades in general mainly the hind feet (Korea J. Immunol. 18, 437, 1996; J. Immunol. 151, 6546,1993).

### EXAMPLE 2

### Preparation of Histone H1

Histone H1 was obtained from Boehringer Mannheim (Catalog Number 223549, lyophilizate, from calf thymus, electrophoretically homogeneous) for the experiment.

### EXAMPLE 3

### Preventive Effect of Histone H1 Against Arthritis

Administration of Histone

As a test group to examine the preventive effect, 1 mg/kg body weight of histone H1 was administered into 10 mice via intraperitoneal injection 2 times every week from the third week (before arthritis induction) up to 10^{th} week after the first injection. Histone H1 was diluted at a concentration of 5 mg/ml in PBS. As a comparison group, 1 mg/kg body weight of dexamethasone, a currently available rheumatoid arthritis drug, was administered into 10 mice via intraperitoneal injection 2 times every week from the third week (before arthritis induction) up to 10^{th} week after the first injection. As a control group, 300 μl of PBS was administered into 20 mice 2 times every week from the third week up to 10^{th} week after the first collagen injection.

Arthritis Induction and Estimation of Arthritis Index

Induction of arthritis was observed 4 weeks after the inoculation of antigens in every group of mice. In the control group that had no treatment after the collagen injection, arthritis induction began 4 weeks after the inoculation (30%). C.I.A. was 64.3% at 5^{th} and 6^{th} weeks and 100% at the 7^{th} week. Compared to this result, the test group of mice that had been injected with histone H1 had a complete prevention of arthritis induction up to the 6^{th} week. C.I.A. in the test group was 60% at 7^{th} and 8^{th} weeks and 80% at the 10^{th} week. The comparison group of mice that had been injected with conventional dexamethasone had 20 to 30% of C.I.A. from 4^{th} to 10^{th} weeks.

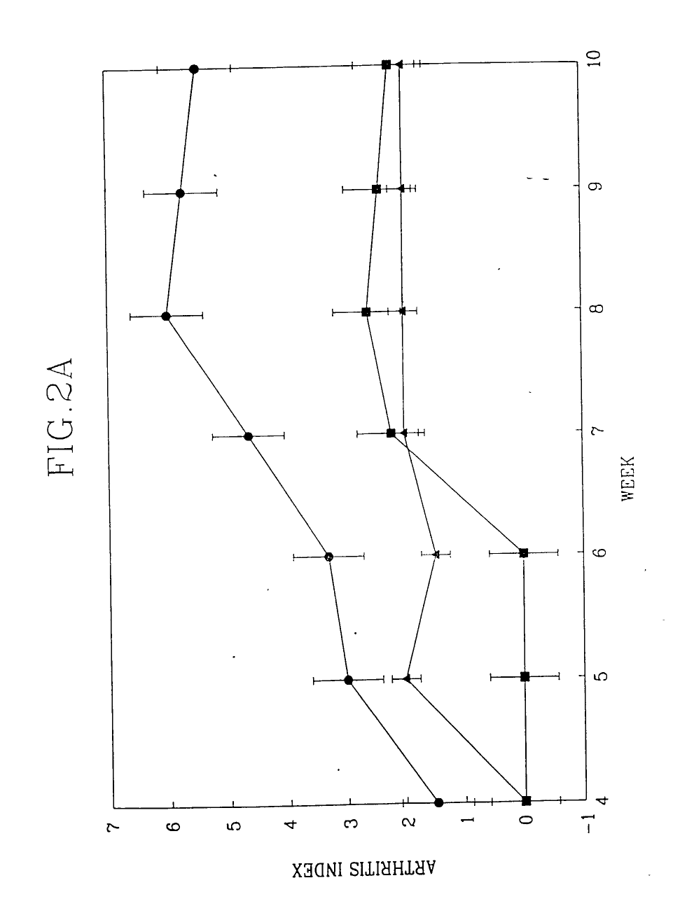

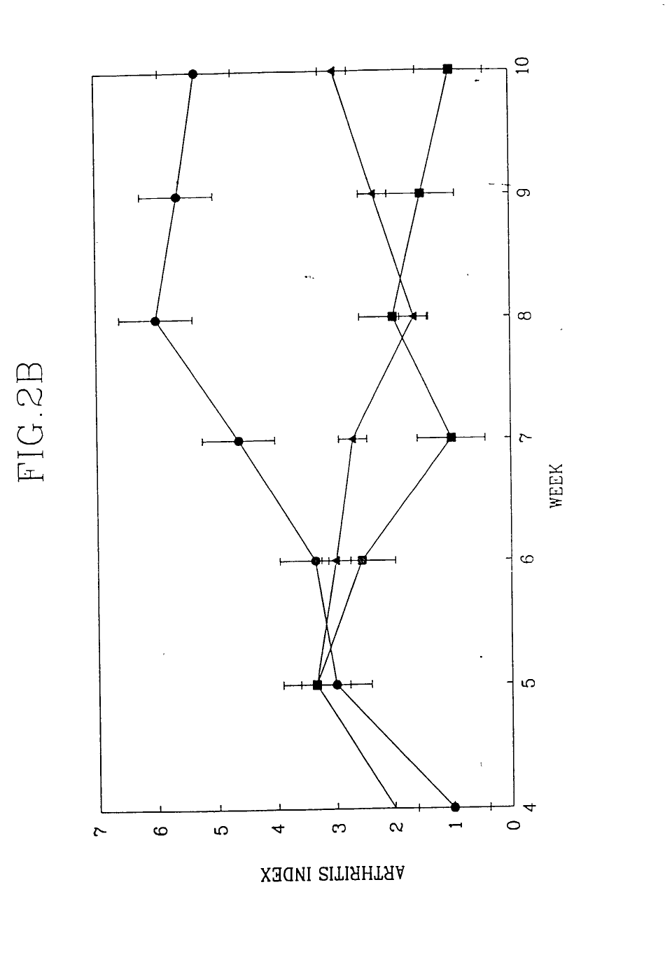

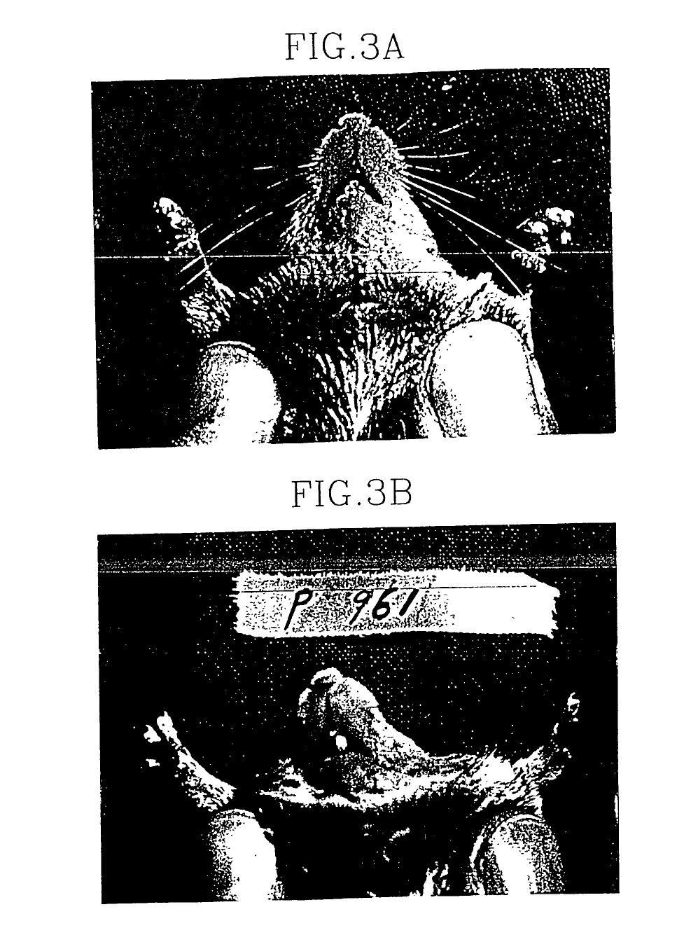

Arthritis index for the comparison group was severe with the values of 1.50±0.55 in 4 weeks, 3.00±1.00 in 5 weeks and had the maximum value of 6.00±2.05 in 8 weeks after the antigen inoculation (FIGS. 2A and 2B). Compared to this result, arthritis in the test group was first observed at the 7^{th} week after the inoculation having 60% of the arthritis index of the control group. In the mice that had arthritis, the arthritis index was ca. half of the control group with the values of 2.67±1.1.5 at 8^{th} weeks and 2.25±1.26 at the 10^{th} week showing that the preventive effect lasts longer than 10 weeks. In the case of dexamethasone injected mice, the arthritis indices were 2.00 and 1.50 at 5 and 6 weeks, respectively showing that the preventive effect is lower than histone administration up to 6 weeks.

Estimation of Immune Reaction: Anti-Collagen Antibody Level

At the 10^{th} week the serum was isolated from the blood obtained through a heart puncture. The serum was kept at −80° C. and thawed immediately before the experiment to measure the anti-collagen antibody level by performing an ELISA (D. E. Tretham & R. A. Dynesius-Trentham, J. Immunol. 130; 2689-2692 (1983)). Type II collagen (25 μg/ml) in 0.1 M PBS was placed in each well of a 96-well polystyrene microplate (Nunc, Denmark) and was incubated at 4° C. for 8 hours. After the incubation, the wells were washed several times with a PBS-0.05% Tween 20 solution. To prevent non-specific immune reactions, PBS-0.5% ovalbumin was added in each well and incubated for an hour at room temperature and subsequently washed again with the PBS-0.05% Tween 20 solution. The serum, diluted 500 times with a buffer solution was added in each well and reacted for 2 hours at room temperature and further washed with the PBS-0.05% Tween 20 solution. After reacting each well with alkaline phosphatase conjugated goat anti-mouse IgA and IgM for 2 hours and adding 1 mg/ml of p-nitrophenyl phosphate, the absorbance at 450 nm was measured. The anti-collagen antibody level was measured twice for each sample and averaged.

The anti-collagen antibody level for the test group was 0.588±.214 (p<0.00005) which was significantly lower than the value of 0.925±075 for the comparison group. The biological significance, however, is not evident since the anti-collagen antibody level was relatively high in every group.

### EXAMPLE 4

### Arthritis Treatment Effect of Histone H1

Administration of Histone H1

As a test group to examine the treatment effect, 1 mg/kg body weight of histone H1 was administered into 10 mice via intraperitoneal injection 2 times every week from the 6^{th} week (after arthritis induction) up to the 10^{th} week. Histone H1 was diluted at a concentration of 5 mg/ml in PBS. As a comparison group, 1 mg/kg body weight of dexamethasone, a currently available rheumatoid arthritis drug, was administered into 10 mice via an intraperitoneal injection 2 times every week from the 6^{th} week (after arthritis induction) up to the 10^{th} week. Identical control group was used as in EXAMPLE 1.

Arthritis Induction and Arthritis Index

C.I.A. in the test group of mice that were treated with histone H1 at the 6^{th} week (after arthritis induction) after antigen inoculation was 37.5% at the 6^{th} week and was reduced to 12.5% at the 7^{th} week. This treatment effect lasted up to the 10^{th} week with C.I.A. of 14.3% at 8^{th} and 10^{th} weeks. In the comparison group that had the dexamethasone treatment, C.I.A. was 22.2%, 33.3% and 22.2% at 7^{th}, 8^{th} and 9^{th} weeks, respectively, showing that the treatment effect was better for histone H1 as a whole.

The arthritis index was 3.33±0.58 at the 5^{th} week after the inoculation for the test group that had the histone treatment. After administration of the histone H1 at the 6^{th} week, the C.I.A remained the same as that at the 5^{th} week however had a reduced arthritis index of 2.67±0.58. After the 7^{th} week, ⅔ of the induced arthritis was completely cured. For the mice that still had the arthritis, the arthritis index decreased from 1.00, 200 to 1.00 at 7^{th}, 8^{th} and 10^{th} weeks, respectively, indicating that histone H1 had a significant treatment effect for rheumatoid arthritis that was already in progress. The arthritis index in the comparison group that had the conventional dexamethasone treatment was 2.67, 1.67 and 3.00 at 7^{th}, 8^{th} and 10^{th} weeks, respectively. (FIGS. 2A and 2B). Pictures of the fore feet of some of the mice were taken at 6^{th} week after the administration of the collagen. Fore feet of the comparison group had edema, one of the symptoms of arthritis, whereas improvement of edema was observed in the test group that had the histone H1 treatment.

Estimation of Immune Reaction: Anti-Collagen Antibody Level

The anti-collagen antibody level was measured to estimate the immune reaction as in EXAMPLE 3. The anti-collagen antibody level for the test group of the treatment effect was 0.540±170 (p<0.00005) which was significantly lower than the value of 0.925±075 for the comparison group. The biological significance, however, was not evident since the anti-collagen antibody level was relatively high in every group.

Pathological Observation by H-E Staining

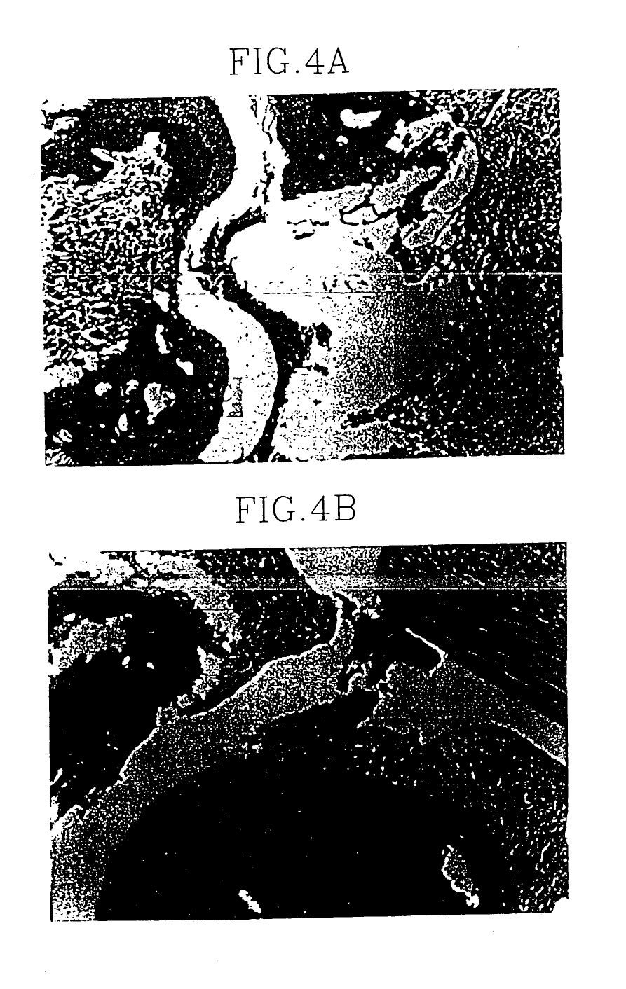

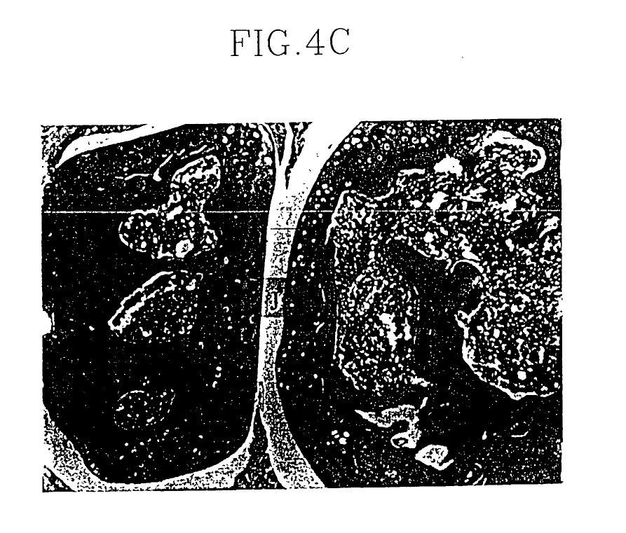

Mice were sacrificed by blood evacuation from the heart. The legs were cut immediately after the sacrifice and fixed in formalin. After the decalcification, legs were stained by hematoxylin-Eosin. The pathological observation by H-E staining showed that the formation of pannus, the erosion of cartilage and the manifestation of inflammatory cells were observed in 10 weeks after the antigen inoculation in the sections of the comparison group (FIGS. 4A and 4B; P=pannus, C=cartilage, J=joint space).

In comparison, the formation of pannus, the erosion of cartilage or the manifestation of inflammatory cells were not observed in 10 weeks showing a normal tissue structure after the antigen inoculation in the section of the test group that had the histone H1 treatment (FIG. 4C).

### EXAMPLE 5

### Preparation of Histone H2A

Histone H2A was obtained from Roche Diagnostics (Catalog Number 1034740, lyophilizate, from calf thymus, electrophoretically homogeneous) for the experiment.

### EXAMPLE 6

### Preparation of an Active Fragment of Histone H2A

An active fragment of histone H2A having the following amino acid sequence was synthesized:

Thr-Arg-Ser-Ser-Arg-Ala-Gly-Leu-Gln-Phe-Pro-Val-Gly-Arg-Val-His-Arg-Leu-Leu-Arg-Lys (SEQ ID NO: 1).

### EXAMPLE 7

### Arthritis Treatment Effect of Histone H2A and its Active Fragment

As a test group to examine the treatment effect, 5 mice for histone H2A and 7mice for histone H2A active fragment were used. 1 mg/kg body weight of histone H2A or histone H2A active fragment was administered into the mice via intraperitoneal injection 2 times every week from the 6^{th} week (after arthritis induction) up to the 10^{th} week. Histone H2A or histone H2A active fragment was diluted at a concentration of 5 mg/ml in PBS. As a comparison group, 1 mg/kg body weight of dexamethasone, a currently available rheumatoid arthritis drug, was administered into 7 mice via an intraperitoneal injection 2 times every week from the 6^{th} week (after arthritis induction) up to the 10^{th} week. As a control group, 300 ml of PBS was administered into 7 mice every week from the third week up to 10^{th} week after the first collagen injection.

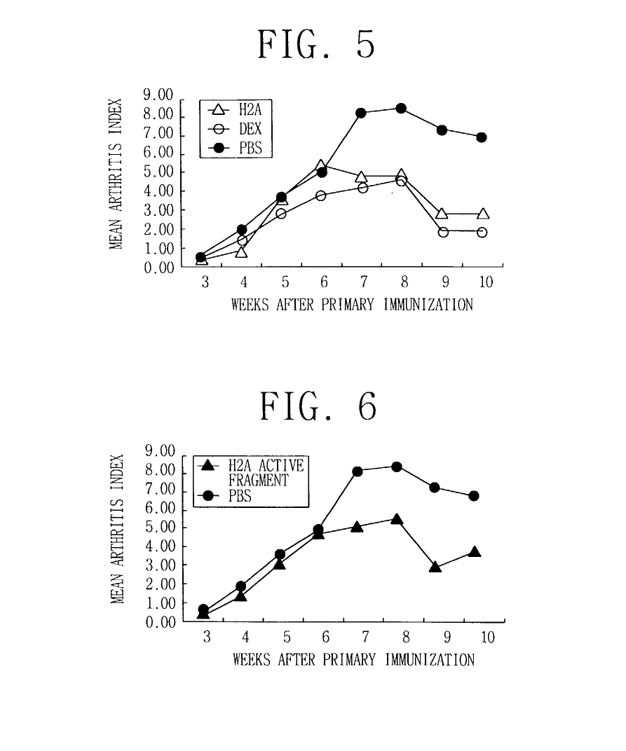

In the test group of mice that were treated with histone H2A, the arthritis index was 3.60±0.55 at the 5^{th} week after the inoculation of collagen. With administration of the histone H2A at the 6^{th} week, the arthritis index of 4.80±0.45 at the 7^{th} week was lower than that of the control group. After the 9^{th} week, the arthritis index was reduced to 2.80 ( FIG. 5).

The arthritis index in the comparison group that had the conventional dexamethasone treatment was 4.57, 1.86 and 1.86 at 8^{th}, 9^{th} and 10^{th} weeks, respectively (FIG. 5).

In the test group of mice that were treated with the active fragment of histone H2A, the arthritis index was 3.14±0.69 at the 5^{th} week after the inoculation of collagen. When the active fragment of histone H2A was administered at the 6^{th} week, the arthritis index of 5.14±0.69 at the 7^{th} week was lower than that of the control group. After the 9^{th} week, the arthritis index was reduced to 3.00 (FIG. 6).

What is claimed is:

1. A method for reducing at least one arthritis symptom in a patient comprising administering to said patient a composition containing a therapeutically effective amount of histone or an active fragment thereof.

2. The method of claim 1 wherein said histone is selected from the group consisting of histone H1, histone H2A, histone H2B, histone H3, histone H4 and a mixture thereof.

3. The method of claim 1 wherein said histone is selected from the group consisting of histone H1, histone H2A and a mixture thereof.

4. A method for preventing rheumatoid arthritis in a patient comprising administering to said patient a composition containing a therapeutically effective amount of histone or an active fragment thereof.

5. The method of claim 4 wherein said histone is selected from the group consisting of histone H1, histone H2A, histone H2B, histone H3, histone H4 and a mixture thereof.

6. The method of claim 4 wherein said histone is selected from the group consisting of histone H1, histone H2A and a mixture thereof.
